(19) United States
(12) Patent Application Publication
(10) Pub. No.: US 2025/0332241 A1
(43) Pub. Date: Oct. 30, 2025

(54) AQUATIC FEED PELLETS AND METHOD OF PREPARATION

(71) Applicant: PRODIGY BIOTECH, INC., West Chester, PA (US)

(72) Inventors: Gopi Kadiyala, Kamataka (IN); Subramanian V. lyer, Royal Palm Beach, FL (US); Satish Chandran, Jupiter, FL (US); Scot Williams, St. Mary's, GA (US)

(73) Assignee: Prodigy Biotech, Inc., West Chester, PA (US)

(21) Appl. No.: 18/869,529

(22) PCT Filed: Jun. 4, 2023
(86) PCT No.: PCT/US2023/067905

§ 371 Date: Nov. 26, 2024

Related U.S. Application Data
(60) Provisional application No. 63/349,314, filed on Jun. 6, 2022.
(60) Provisional application No. 63/499,011, filed on Apr. 28, 2023.

(51) Int. Cl. A61K 39/12; A23K 10/20; A23K 20/137; A23K 20/163; A23K 40/20; A23K 50/80; A61K 39/00; A61K 39/42; A61K 47/36

(52) U.S. Cl. CPC A61K 39/12; A23K 10/20; A23K 20/137; A23K 20/163; A23K 40/20; A23K 50/80; A61K 39/42; A61K 47/36; A61K 2039/542

(57) ABSTRACT

Methods of producing feed pellets containing antiviral proteins for protecting aquatic animals against viruses by passive immunization are disclosed. The pellets have an effective amount of an active antiviral protein incorporated into basal feed stock along with stabilizing. gelling and bioavailability enhancing agents. The antiviral proteins are IgY antibodies contained in hyperimmune egg powder obtained from eggs produced by avian species exposed to antigens associated with viral infections. To protect shrimp against white spot syndrome virus (WSSV) the antigen consists of a protein made from the effective parts of the VP28 and VP19 sequences of the WSSV. In one embodiment, the stabilizing agent is sodium alginate, the gelling agent is carboxy methyl cellulose and the bioavailability enhancing agent is piperine. In one embodiment the active protein and associated supplements are incorporated throughout the feed pellet. while in another embodiment they are sprayed onto the surface of the feed pellet.

### CROSS-REFERENCE TO RELATED APPLICATIONS

This is a PCT patent application claiming priority to U.S. provisional patent application No. 63/349,314 that was filed on Jun. 6, 2022, entitled “Anti-viral Aquatic Feed Pellets”, and to U.S. provisional patent application No. 63/499,011 that was filed on Apr. 28, 2023, entitled “Aquatic Feed Pellets”, the contents of both of which are hereby fully incorporated by reference.

### TECHNICAL FIELD

The invention relates to methods of producing and using feed pellets containing active proteins for protecting aquatic populations by passive immunization, and more particularly to producing and using feed pellets containing IgY antibodies for protecting shrimp against white spot shrimp virus (WSSV) by passive immunization.

### BACKGROUND ART

Aquaculture plays a vital role in meeting the growing demand for seafood. It provides a sustainable and reliable source of protein-rich food, reducing the pressure on wild fish populations. There are, however, many challenges associated with aquaculture, not the least of which is disease management.

Shrimp farming, particularly of the Pacific white shrimp (Litopenaeus vannamei), is a major aquaculture industry globally with significant activity in places such as, but not limited to, China, Thailand, Indonesia, India, Ecuador, and Vietnam.

Shrimps are, however, susceptible to white spot syndrome virus (WSSV), a highly contagious and devastating viral disease. It is one of the most significant viral pathogens in the aquaculture industry, causing severe economic losses. Outbreaks can lead to massive mortality rates, resulting in financial losses for farmers and the wider aquaculture sector. The disease has been responsible for large-scale shrimp farming failures in various countries.

Antiviral proteins have been developed that have shown to be effective against WSSV when injected into shrimp, as detailed in, for instance, patent publication WO/2003/070258 filed on behalf of Lee & Joe Biotech Co. of Korea on Feb. 2, 2003 entitled “Anti-White Spot Syndrome Virus Igy” that describes a “a yolk antibody against the shrimp white spot virus, and more particularly, to an egg discharged from immunized animals into which the shrimp white spot virus or its proteins are injected, a yolk antibody isolated from the egg, and a composition for control against shrimp white spot virus infection comprising the yolk antibody. The yolk antibody against the shrimp virus of the invention acts on the shrimp white spot virus to suppress its infectivity, and accordingly it can be used as a preventive agent against shrimp virus infection”.

However, delivering antiviral proteins via aquatic feedstocks can pose several challenges due to the unique characteristics of aquatic environments and the nature of the proteins. These difficulties include factors such as, but are not limited to, stability, protein digestion, bioavailability and absorption.

What is desired is the development of aquatic feed pellets that can incorporate an appropriate antiviral protein and deliver it effectively to creatures such as, but not limited to, shrimp.

### DISCLOSURE OF INVENTION

Inventive feed pellets containing antiviral proteins for protecting aquatic animals against viruses by passive immunization, and the methods of producing them, are disclosed.

In a preferred embodiment, an aquatic feed pellet that may incorporate an effective amount of an active protein may be made up of a basal feed stock supplemented by compounds such as, but not limited to, a stabilizing agent, a gelling agent and a bioavailability enhancing agent.

The basal feed stock may, for instance, be a commercially available feed stock containing ingredients such as, but not limited to, carbohydrates, protein, fat, and fiber.

The stabilizing agent may, for instance, function to improve shelf life of the feed pellets by inhibiting the growth of microorganisms and preventing oxidation that may lead to spoilage of the feed pellets. A suitable stabilizing agent may, for instance, be sodium alginate.

The gelling agent may function, for instance, to bind the ingredients together and create a more cohesive pellet that may be less likely to break apart or crumble during transportation, storage, or handling. One suitable gelling agent may be carboxy methyl cellulose.

The bioavailability enhancing agent may, for instance, function to improve the availability and absorption of the active protein by the animal's digestive system. A suitable bioavailability enhancing agent may, for instance, be piperine.

The active protein may, for instance, be a substance such as, but not limited to, hyperimmune egg powder. The hyperimmune egg powder may contain IgY antibodies and may be obtained from eggs produced by an egg laying avian species, such as chickens, that may have been exposed to viral proteins associated with a virus threating an aquatic animal, such as, but not limited to, the WSSV threating shrimps.

In one embodiment, the active protein and associated supplements may be incorporated throughout the feed pellet. In another embodiment, the active protein and associated supplements may be sprayed onto the surface of the feed pellet.

These embodiments are described in more detail below.

### BRIEF DESCRIPTION OF DRAWINGS

FIG. 1 is a flow diagram showing representative steps of providing aquatic feed pellets of the present invention.

FIG. 2 is a flow diagram showing representative steps of one method of providing aquatic feed pellets of the present invention.

FIG. 3 is a flow diagram showing representative steps of a further method of providing aquatic feed pellets of the present invention.

FIG. 4 is a flow diagram showing representative steps of one method of obtaining hyperimmune egg powder of the present invention.

FIG. 5 is a flow diagram showing representative steps of adding hyperimmune egg powder to aquatic pellets of the present invention.

FIG. 6 is a flow diagram showing representative steps of yet a further method of providing aquatic feed pellets of the present invention.

FIG. 7 is a flow diagram showing representative steps of yet another method of providing aquatic feed pellets of the present invention.

### BEST MODE FOR CARRYING OUT THE INVENTION

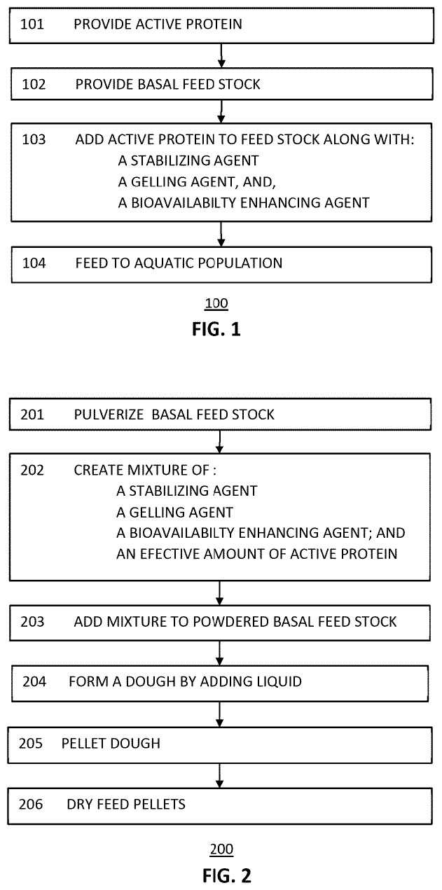

FIG. 1 is a flow diagram 100 showing representative steps of providing aquatic feed pellets of the present invention.

In Step 101 “PROVIDE ACTIVE PROTEIN” a suitable active protein may be produced or procured. For instance, the active protein may, for instance, be a protein such as, but not limited to, an IgY antibody that may be contained in a hyperimmune egg powder. A method of by which such an antibody may be produced is detailed below.

In Step 102 “PROVIDE BASAL FEED STOCK” a basal feed stock suitable for providing nutrition to an aquatic population may be produced or procured. Such a feed stock may include suitable amounts of carbohydrates, protein, fat, and fiber. Commercially available aquatic feed stock may, for instance, be obtained from companies such as, but not limited to, Charoen Pokphand Co. Ltd., of Thailand. Such a commercially available feed stock typically contains about 38% protein, 5% fat, and less than 3% fiber.

In Step 103 “ADD ACTIVE PROTEIN TO FEED STOCK ALONG WITH: A STABILIZING AGENT, A GELLING AGENT, AND A BIOAVAILABILTY ENHANCING AGENT” the feed stock may be augmented by other ingredients such as, but not limited to, one or more stabilizing agents, one or more gelling agents, and one or more bioavailability enhancing agents.

Stabilizing agents may be added to animal feed stock pellets to improve their shelf life. A primary function of a stabilizing agent may be to inhibit the growth of microorganisms and prevent chemical reactions that can lead to degradation of the feed.

Stabilizing agents include antioxidants such as, but not limited to, vitamin E and BHT (butylated hydroxytoluene), preservatives such as, but not limited to, propionic acid and sorbic acid, and acidifiers such as, but not limited to, citric acid and lactic acid.

Another a stabilizing agent used in animal feed is sodium alginate, a natural polymer derived from brown seaweed.

Sodium alginate may be particularly useful in aquatic animal feeds, as it may help to improve the water stability of the pellets, and prevent them from dissolving or breaking apart too quickly in water. This may help to ensure that the feed remains accessible and available to the animals for longer periods of time, and may reduce waste.

Gelling agents may be used in animal feed stock pellets. Their primary function may be to bind the ingredients together and create a more cohesive pellet.

Gelling agents used in animal feed stock pellets include natural binders such as, but not limited to, gelatin, starch, and pectin, as well as synthetic binders such as, but not limited to, methylcellulose and carboxy methyl cellulose.

Bioavailability enhancing agents may be used in animal feed stock pellets to improve the availability and absorption of nutrients by the animal's digestive system. Bioavailability enhancing agents include enzymes, organic acids, probiotics, and prebiotics.

Piperine is one particularly effective bioavailability enhancing agent and has been found to increase bioavailability of different drugs by amounts ranging from 30% to 200%.

In Step 104 “FEED TO AQUATIC POPULATION” the basal stock, to which the stabilizing agent, the gelling agent, the bioavailability enhancing agent and the active protein have been added, may then be fed to an aquatic population.

The result of feeding trials using aquatic feed stock of the present invention are detailed in, for instance, U.S. provisional patent application No. 63/349,314 that was filed on Jun. 6, 2022, entitled “Anti-viral Aquatic Feed Pellets” and, U.S. provisional patent application No. 63/499,011 that was filed on Apr. 28, 2023, entitled “Aquatic Feed Pellets”, the contents of both of which are hereby fully incorporated by reference.

FIG. 2 is a flow diagram 200 showing representative steps of one method of providing aquatic feed pellets of the present invention.

In Step 201 “PULVERIZE BASAL FEED STOCK” a suitable aquatic feed stock may be reduced to a powder. The feed stock may, for instance, be a commercially available feed stock as provided by, for instance, Charoen Pokphand Co. Ltd., Bangkok, Thailand, and have suitable amounts of ingredients such as, but not limited to, carbohydrates, protein, fat, and fiber. Such a commercially available feed stock typically contains about 38% protein, 5% fat, and less than 3% fiber.

In Step 202 “CREATE MIXTURE OF: A STABILIZING AGENT, A GELLING AGENT, A BIOAVAILABILTY ENHANCING AGENT; AND AN EFFECTIVE AMOUNT OF ACTIVE PROTEIN, a mixture may be created. This mixture may be comprised of any of the appropriate ingredients detailed above. However, in a preferred embodiment, the stabilizing agent may be sodium alginate, the gelling agent may be carboxy methylcellulose, the bioavailability enhancing agent may be piperine, and the active protein may hyperimmune egg powder which may contain IgY antibodies.

The proportions of the ingredients may vary. In a representative formulation sodium alginate may be present in a range of 20 to 90 grams per kilogram of feed stock, carboxy methyl cellulose in a range of 5 to 15 grams per kilogram, piperine in a range of 0.1 to 1 grams per kilogram, and, hyperimmune egg powder in a range of 0.1 to 10 grams per kilogram.

However, in a more preferred embodiment, the ingredients in the mixture may be present in the following amounts: sodium alginate in a range of 40 to 60 grams per kilogram of feed stock, carboxy methyl cellulose in a range of 8 to 12 grams per kilogram, piperine in a range of 0.2 to 1 gram per kilogram, and, hyperimmune egg powder in a range of 0.25 to 5 grams per kilogram.

In a most preferred embodiment, the ingredients may consist of the following amounts: sodium alginate 50 grams per kilogram of feed stock, carboxy methyl cellulose 9.5 grams per kilogram, piperine 0.5 grams per kilogram, and hyperimmune egg powder 2.5 grams per kilogram.

In Step 203 “ADD MIXTURE TO POWDERED BASAL FEED STOCK” the mixture obtained in Step 202 may be added to the pulverized feed stock.

In Step 204 “FORM A DOUGH BY ADDING LIQUID”, the mixture and pulverized feed stock may be formed into a dough by adding a suitable amount of a suitable liquid. In a preferred embodiment, the liquid may be water and the quantities detailed in Step 202 may be added to a liter of the water. In a more preferred embodiment, the liquid may be distilled water.

In Step 205 “PELLET DOUGH” the dough obtained in Step 204 may be formed into suitable sized feed pellets. This may, for instance, be done by forcing the dough through suitably sized apertures. This may, for instance, be done using any suitable commercially available stainless steel kitchen press using, for instance, a steel mesh having a pore size of 2 mm. For larger batch sizes, a commercial pellet mill may be used, such as, but not limited to, a CPM Pellet Mill manufactured by CPM (California Pellet Mill) of Blaine, MN. Their system utilizes a die and roller system to compress the raw materials into pellets of uniform size and density.

In Step 206 “DRY FEED PELLETS”, the feed pellets obtained in Step 205 may be dried so as to be stored and/or fed to an aquatic population. Drying may, for instance, be done overnight at room temperature, or at 37 degrees Centigrade for 3 hours. Storing may be done at ambient room temperature as is typical in the industry.

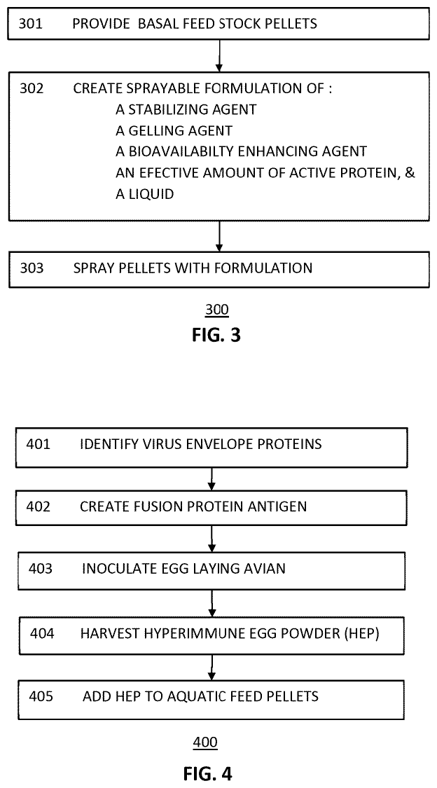

FIG. 3 is a flow diagram 300 showing representative steps of a further method of providing aquatic feed pellets of the present invention.

In Step 301 “PROVIDE BASAL FEED STOCK PELLETS” a suitable aquatic feed stock may be provided in pellet form. The composition of those pellets may, for instance, be the same as or similar to commercially available aquatic feedstocks. Typically, such aquatic feed stocks contain protein in a range of 35% to 45% by weight, and fat in a range of 3% to 8% by weight.

In Step 302 “CREATE SPRAYABLE FORMULATION OF: A STABILIZING AGENT, A GELLING AGENT, A BIOAVAILABILTY ENHANCING AGENT, AN EFFECTIVE AMOUNT OF ACTIVE PROTEIN, & A LIQUID” a sprayable formulation may be created. This sprayable formulation may be comprised of any of the appropriate ingredients detailed above. However, in a preferred embodiment, the stabilizing agent may be sodium alginate, the gelling agent may be carboxy methylcellulose, the bioavailability enhancing agent may be piperine, and the active protein may hyperimmune egg powder which may contain IgY antibodies.

The proportions of the ingredients may vary. For instance, a sprayable formulation suitable for spray coating approximately 1 kg of feed pellets may contain sodium alginate in a range of 20 to 60 milligrams, carboxy methyl cellulose in a range of 5 to 45 milligrams, piperine in a range of 2 to 20 milligrams, and hyperimmune egg powder in a range of 200 to 750 milligrams.

However, in a more preferred embodiment, a sprayable formulation suitable for spray coating approximately 1 kg of feed pellets may be made up of sodium alginate in an amount in a range of 30 to 50 milligrams, carboxy methyl cellulose in an amount in a range of 10 to 30 milligrams, piperine in an amount in a range of 5 to 15 milligram, hyperimmune egg powder in an amount in a range of 400 to 550 milligrams all dissolve in approximately 20 milliliters of water. The water may preferably be distilled.

In a most preferred embodiment, the sprayable formulation for 1 kg of feed stock pellets may be made up of the ingredients in the following amounts: 40 mg of sodium alginate, 20 mg of carboxy methyl cellulose, 10 mg of piperine and 500 mg of hyperimmune egg powder, all dissolved in approximately 20 milliliters of water.

In Step 303 “SPRAY PELLETS WITH FORMULATION” the aquatic feed stock pellets may be surface coated by, for instance, spray coating, using the formulation obtained in Step 302. This coating may be done with any suitable surface coating machinery, such as, but not limited to a Continuous Pellet Coater (CPC) as supplied by Van Aarsen International of Panheel in the Netherlands.

FIG. 4 is a flow diagram 400 showing representative steps of obtaining hyperimmune egg powder of the present invention.

In Step 401 “IDENTIFY VIRUS ENVELOPE PROTEINS” the envelope proteins of a virus threatening a bottom feeding aquatic species may be identified. A reason for identifying envelope proteins may be that these are the proteins that are typically exposed when the virus enters the aquatic species. Moreover, the envelope proteins typically play an important role in a virus attaching to, and entering, the cells of the aquatic species. If antibodies can be developed that bind to these proteins, and those antibodies administered to the aquatic species, they may be effective in preventing the virus from entering the species cells.

White spot disease (WSD) is a highly contagious viral infection of decapod crustaceans that can cause high levels of mortality in cultured shrimp. Since its first outbreak in 1992-93, this disease has caused serious economic losses. The causative agent of WSD is white spot syndrome virus (WSSV), which is a large, nonoccluded, enveloped, rod-shaped to elliptical DNA virus with a tail-like extension at one end. WSSV multiplies in the nucleus and has a very broad host range among crustaceans. Most of the predicted open reading frames in its genome (˜300 kilobase pairs (kbp)) encode polypeptides that show no homology to known proteins, while the identifiable genes are mainly involved in nucleotide metabolism and DNA replication.

However, two of the viral particles (VP), VP28 and VP19 are the most exposed and abundant in the WSSV envelope and thus likely to be the first to come in contact with host cells. VP28 appears to play a key role in the initial steps of the systemic WSSV infection in shrimp. VP28 and VP19 are both located in the WSSV envelope. These proteins contain hydrophobic regions, which may have a function in anchoring these proteins in the envelope.

In Step 402 “CREATE FUSION PROTEIN ANTIGEN” a single fusion protein may be created as an antigen. This fusion protein may, for instance, be made up of the effective elements of the viral particle proteins found to be envelope proteins in a virus threatening an aquatic animal. Creating a single fusion protein may reduce the cost of subsequent laboratory or industrial replication of the protein.

A suitable fusion protein to be used as an antigen for WSSV may, for instance, be made up of the effective parts of VP28 and VP19 to produce a single, truncated fusion viral protein TrVP29:19. The DNA sequence necessary for the fusion protein may be produced using standard DNA synthesizing techniques and technology. After PCR amplification, the DNA may, for instance, be inserted into a suitable expression vector to produce the protein antibodies. These synthetic TrVP29:19 protein antibodies may then be harvested from the expression vector. Details of suitable techniques are published in, for instance, PCT publication WO 03/070258 entitled “Anti-White Spot Syndrome Virus IGY” by Jong-Hwa Lee et. al, and in the paper “Shrimp protected from WSSV disease by treatment with egg yolk antibodies (IgY) against a truncated fusion protein derived from WSSV” published in Aquaculture Volume 237, issues 1-4, 2 Aug. 2002, pages 21-31 by D. K. Kim et. al, the contents of which are hereby incorporated by reference.

In Step 403 “INOCULATE EGG LAYING AVIAN” a suitable, egg laying member of an avian species may be inoculated with the fusion antigen protein obtained in the previous step. Female chickens may, for instance, be suitable egg laying members of an avian species.

In Step 404 “HARVEST HYPERIMMUNE EGG POWDER (HEP)” hyperimmune egg powder may be harvested from eggs produced by the inoculated egg laying avians. The yolks of these eggs typically contain IgY antibodies. These eggs may, for instance, be collected, their yolk separated out and dried to form hyperimmune egg powder. In a further embodiment, the IgY antibody proteins may be separated out from the other proteins present in the egg yolk.

In Step 405 “ADD HEP TO AQUATIC FEED PELLETS” hyperimmune egg powder containing the IgY antibody proteins harvested in the previous step may be added to aquatic feed pellets. These feed pellets may then be fed to the threatened aquatic species who, on ingesting the antibody laden feed pellets may be effectively immunized against the virus.

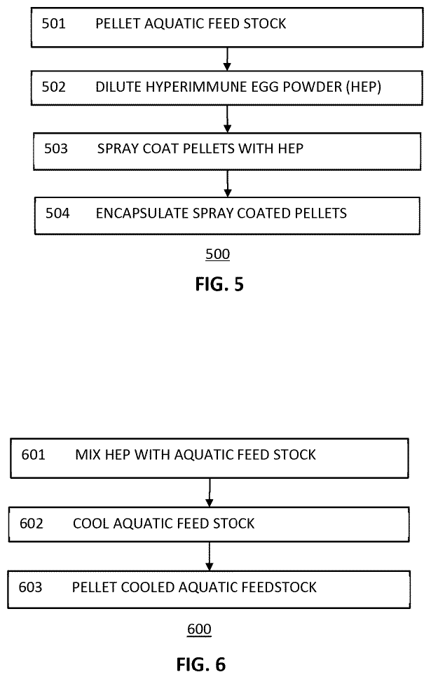

FIG. 5 is a flow diagram 500 showing representative steps of adding hyperimmune egg powder to aquatic pellets of the present invention.

In Step 501 “PELLET AQUATIC FEED STOCK”, aquatic feed stock may be pelleted using one of the well-known pelleting technologies such as, but not limited to, pellet extruders and pellet mills.

Aquatic feed stock is typically a combination of finely ground protein matter such as, but not limited to, dry fish, prawn head waste, squilla and squid, and added fish oil. For bottom feeding aquatic animals, the feed pellets are preferably denser than water and resistant to dissolving in water. Drying the aquatic feed stock such that it contains less than 10% of water, mixing it with low oil content grain flour and finely grinding it to have particles that are 200-300 microns in diameter may enhance pellet forming and produce pellets that are easily digested.

The hard, dense pellets preferred for bottom feeding aquatic species may most reliably be produced using pellet mills. These may compact the feed stock into cylindrical pellets have a range of sizes from 0.6 to 3 mm. During the pelleting process, the aquatic feed stock may be extruded through dies. This extrusion may heat the aquatic feed stock to as much as 100-130 degrees C., enough to cook proteins and gelatinize starch.

In Step 502 “DILUTE HYPERIMMUNE EGG POWDER (HEP)” the harvested hyperimmune egg powder containing IgY antibodies may be diluted in preparation for spray coating extruded pellets. This dilution may, for instance, be with water.

In Step 503 “SPRAY COAT PELLETS WITH HEP” the diluted mixture of hyperimmune egg powder containing the IgY antibodies may be spray coated onto the pelleted feed stock. By applying the hyperimmune egg powder containing the IgY antibodies after the pelleting process, the heating during extrusion may be avoided, thereby avoiding the denaturing the IgY antibody proteins.

In Step 504 “ENCAPSULATE SPRAY COATED PELLETS” the feed pellets coated with hyperimmune egg powder containing IgY antibodies may be encapsulated. This may be necessary to avoid the hyperimmune egg powder containing the IgY antibodies being dissolved away from the feed pellets when they are submerged in water for feeding the aquatic species. Suitable encapsulation materials may include edible drying oils such as, but not limited to, linseed oil and poppy seed oil. The encapsulation may, for instance, be accomplished using a form of fluid bed coating.

FIG. 6 is a flow diagram 600 showing representative steps of yet a further method of providing aquatic feed pellets of the present invention.

In Step 601 “MIX HEP WITH AQUATIC FEED STOCK” the hyperimmune egg powder containing IgY antibodies may be mixed with an aquatic feed stock.

Step 602 “COOL AQUATIC FEED STOCK”. In pellet mills, the feed stock may be mixed in a feeder as it is driven toward the feed hopper and die cavity by a helical screw. Conventionally, this mixing is accompanied by steam to soften the feed. However, when the feed stock is pressed through the dies to form pellets, the compression may raise the temperature of the feed stock to as much as 100-130 degrees C., enough to denature the IgY antibodies contained in the hyperimmune egg powder. To obviate this, instead of mixing in the feeder using steam, the mixing may be accomplished using cooled, humid air. The cooling may, for instance, be done by mixing vapor from boiling liquid nitrogen into the feeder. In this way, the feedstock may be mixed at a sufficiently low temperature such that when it is pressed through the dies, the temperature rise may be insufficient to denature the IgY antibodies contained in the hyperimmune egg powder.

In Step 603 “PELLET COOLED AQUATIC FEEDSTOCK” the cooled aquatic feedstock may be compressed through suitably sized dies in order to form dense, hard feed pellets suitable for feeding to bottom feeding aquatic creatures such as, but not limited to, shrimp. Moreover, the IgY antibodies in the hyperimmune egg powder may not be denatured by such a process and may be active in the feed pellets, thereby allowing for passive immunization of aquatic creatures such as, but not limited to, shrimp that eat the feed pellets.

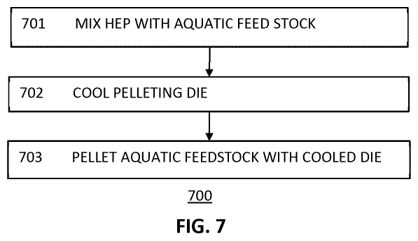

FIG. 7 is a flow diagram 700 showing representative steps of yet another method of providing aquatic feed pellets of the present invention.

In Step 701 “MIX HEP ANTIBODIES WITH AQUATIC FEED STOCK” the hyperimmune egg powder (HEP) that may contain IgY antibodies may be mixed with a suitable aquatic feed stock.

In Step 702 “COOL PELLETING DIE” the pellet forming dies may be cooled. When the feed stock is pressed through the dies to form pellets using conventional dies, the compression may raise the temperature of the feed stock to as much as much as 100-130 degrees C., enough to denature the IgY antibodies contained in the hyperimmune egg powder. To obviate this, the dies themselves may be cooled. If the dies are cooled to a sufficiently low temperature, when the feedstock is pressed through the dies it will also be cooled sufficiently that the compression induced temperature rise is insufficient to denature the IgY antibodies contained in the hyperimmune egg powder. The cooling may, for instance, be accomplished using vapor from boiling liquid nitrogen.

In Step 703 “PELLET AQUATIC FEEDSTOCK WITH COOLED DIE” the aquatic feedstock may be compressed through suitably sized and cooled dies in order to form dense, hard feed pellets suitable for feeding to bottom feeding aquatic creatures such as, but not limited to, shrimp. Moreover, the IgY antibodies may not be denatured by such a process and may be active in the feed pellets, thereby allowing for passive immunization of aquatic creatures such as, but not limited to, shrimp that eat the feed pellets.

In a further embodiment of the method of producing feed pellets for protecting bottom feeding aquatic animals against viruses of this invention, Steps 602 and 702 may combined, i.e., the feed stock may be cooled, and the pelleting dies may also be cooled.

### INDUSTRIAL APPLICABILITY

The present inventions may have applicability in the field of aquiculture, including in the field of shrimp production.

1-11. (canceled)

12. An aquatic feed pellet comprising:
a basal feed stock;
a stabilizing agent;
a gelling agent;
a bioavailability enhancing agent; and
IgY antibodies.

13. The aquatic feed pellet of claim 12, wherein the stabilizing agent, the gelling agent, the bioavailability enhancing agent, and the IgY antibodies are distributed throughout the feed pellet.

14. The aquatic feed pellet of claim 12, wherein the IgY antibodies are comprised within a hyperimmune egg powder.

15. The aquatic feed pellet of claim 12, wherein, the IgY antibodies are obtained by:
identifying at least two viral proteins associated with an envelope of a virus threating an aquatic animal;
creating an antigen comprising a fusion protein comprising effective elements of the two viral proteins;
inoculating one or more egg laying member of an avian species with the antigen; and
obtaining the IgY antibodies to the antigen from one or more eggs produced by one or more of the egg laying avian species.

16. (canceled)

17. The aquatic feed pellet of claim 12, wherein, the stabilizing agent, the gelling agent, the bioavailability enhancing agent, and the IgY antibodies are coated onto the feed pellet.

18. The aquatic feed pellet of claim 12, wherein,
the stabilizing agent is sodium alginate;
the gelling agent is carboxy methyl cellulose; and
the bioavailability enhancing agent is piperine.

19. (canceled)

20. The aquatic feed pellet of claim 18, wherein, for 1 kg of the basal feed stock,
the sodium alginate is in an amount in a range of 30 to 50 milligrams;
the carboxy methyl cellulose is in an amount in a range of 10 to 30 milligrams;
the piperine is in an amount is in a range of 5 to 15 milligrams; and
the IgY antibodies are comprised within a hyperimmune egg powder, and the hyperimmune egg powder is in an amount in a range of 400 to 550 milligrams.

21. The aquatic feed pellet of claim 18, wherein, for 1 kg of basal feed powder, the sodium alginate is in an amount in a range of 40 to 60 grams;
the carboxy methyl cellulose is in an amount in a range of 8 to 12 grams;
the piperine is in an amount in a range of 0.2 to 1 gram; and
the IgY antibodies are comprised within a hyperimmune egg powder, and the hyperimmune egg powder is in an amount in a range of 0.25 to 5 grams.

22. The aquatic feed pellet of claim 12, wherein the stabilizing agent is sodium alginate.

23. The aquatic feed pellet of claim 12, wherein the gelling agent is carboxy methyl cellulose.

24. The aquatic feed pellet of claim 12, wherein the bioavailability enhancing agent is piperine.

25. The aquatic feed pellet of claim 12, wherein the IgY antibodies are IgY antibodies to one or more white spot syndrome virus (WSSV) proteins.

26. The aquatic feed pellet of claim 23, wherein the one or more WSSV proteins are VP28 and VP19.

27. The aquatic feed pellet of claim 15, wherein the virus is white spot syndrome virus (WSSV).

28. A method for preventing or treating WSSV infection in an aquatic species, comprising orally administering to the species a therapeutically effective amount of a hyperimmunized egg product obtained from an egg-producing animal, thereby preventing or treating the WSSV infection in the species, wherein the hyperimmunized egg product comprises a therapeutically effective amount of one or more antibodies to an antigen selected from the group consisting of VP19 and VP28.

29. The method of claim 28, wherein the hyperimmunized egg product comprises a therapeutically effective amount of one or more antibodies to VP19, and a therapeutically effective amount of one or more antibodies to VP28.

30. The method of claim 28, wherein the hyperimmunized egg product is whole egg.

31. The method of claim 28, wherein the hyperimmunized egg product is hyperimmune egg powder.

32. The method of claim 28, wherein the hyperimmunized egg product is comprised within an aquatic feed pellet.

33. The method of claim 32, wherein the aquatic feed pellet further comprises:
a basal feed stock;
a stabilizing agent;
a gelling agent; and
a bioavailability enhancing agent.

34. The method of claim 33, wherein the stabilizing agent is sodium alginate, the gelling agent is carboxy methyl cellulose, and the bioavailability enhancing agent is piperine.
